(19) United States
(12) Patent Application Publication
(10) Pub. No.: US 2024/0050398 A1
(43) Pub. Date: Feb. 15, 2024

(54) METHOD AGAINST SNAKE ENVENOMATION

(71) Applicants: UBI PHARMA INC., Hukou Township (TW); National Tsing Hua University, Hsinchu City (TW)

(72) Inventors: Wen-Guey WU, Hsinchu City (TW); Li-Kin HUANG, Hsinchu City (TW); I-Jin LIN, Hsinchu City (TW)

(21) Appl. No.: 18/447,057

(22) Filed: Aug. 9, 2023

Related U.S. Application Data
(60) Provisional application No. 63/397,147, filed on Aug. 11, 2022.

(51) Int. Cl. A61K 31/352; A61P 39/02

(52) U.S. Cl. CPC A61K 31/352; A61P 39/02

(57) ABSTRACT

A method against snake envenomation includes administering to a subject in need thereof a pharmaceutical composition containing a flavonoid compound. The flavonoid compound is selected from the group consisting of isorhamnetin, quercetin, and a combination thereof.

### CROSS-REFERENCE TO RELATED APPLICATION

This application claims the benefit of U.S. Provisional Patent Application No. 63/397,147, filed on Aug. 11, 2022, which is incorporated by reference herein in its entirety.

### FIELD

The disclosure relates to a method against snake envenomation using a pharmaceutical composition containing a flavonoid compound.

### BACKGROUND

Snake venom is an extremely toxic saliva that may be found in various snake species and mainly contains numerous kinds of proteins and enzymes responsible for its toxin, and may be generally classified into two types, both having a high likelihood of being lethal to a patient afflicted therewith. One of the types is a hemotoxic venom that may exist in a Protobothrops spp. such as Protobothrops mucrosquamatus, and another type is a neurotoxic venom that may come from a Naja spp., such as Naja nivea and Naja atra. When administering emergency treatment, a medical professional may need to search for a corresponding antivenom based on the type of the snake venom that the patient suffered from and inject the antivenom into the patient as soon as possible. However, the time of transporting the patient to a hospital and determining which type of the snake venom the patient encountering, and the difficulty of storing the antivenom are all clinical challenges for such emergency treatment. Accordingly, those skilled in the art still strive to develop a new strategy that is more effective against snake envenomation.

Isorhamnetin and quercetin are two kinds of flavonoid compounds found in medicinal plants such as sea buckthorn (Hippophae rhamnoides L.), and are currently known to have anti-tumor as well as anti-adipogenesis effects, and also be capable of treating enteritis. However, it remains unknown whether isorhamnetin and quercetin are effective against snake envenomation.

### SUMMARY

Therefore, an object of the disclosure is to provide a method against snake envenomation, which can alleviate at least one of the drawbacks of the prior art, and which includes administering to a subject in need thereof a pharmaceutical composition containing a flavonoid compound. The flavonoid compound is selected from the group consisting of isorhamnetin, quercetin, and a combination thereof.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 shows the viability percentage determined in each of a pathological control group and experimental groups 1 and 2 of Example 1, infra.

FIG. 2 shows the viability percentage determined in each of pathological control group 3 and experimental group 3 of Example 1, infra.

FIG. 3 shows the viability percentage determined in each of pathological control group 4 and experimental group 4 of Example 1, infra.

FIG. 4 shows the viability percentage determined in each of pathological control group 5 and experimental group 5 of Example 1, infra.

FIG. 5 shows the viability percentage determined in each of pathological control group 6 and experimental group 6 of Example 1, infra.

FIG. 6 shows the viability percentage determined in each of pathological control group 7 and experimental group 7 of Example 1, infra.

FIG. 7 shows the area of cutaneous ulcers determined in each of pathological control group and experimental groups 1 and 2 of Example 2, infra.

FIG. 8 shows the area of cutaneous ulcers determined in each of pathological control group 3 and experimental group 3 of Example 2, infra.

### DETAILED DESCRIPTION

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless otherwise defined, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

In the development of methods against snake envenomation, the applicants unexpectedly found that both isorhamnetin and quercetin, which are two kinds of flavonoid compounds, are capable of preventing death and alleviating tissue necrosis caused by various snake venoms (of different venomous snakes), On the other hand, the applicants also found that, apigenin is not capable of delivering such effects in preventing death, it may even accelerate death despite having a similar structure to those of isorhamnetin and quercetin. Therefore, isorhamnetin and quercetin are expected to be effective against snake envenomation.

Accordingly, the disclosure provides a method against snake envenomation, which includes administering to a subject in need thereof a pharmaceutical composition containing a flavonoid compound. The flavonoid compound is selected from the group consisting of isorhamnetin, quercetin, and a combination thereof. In certain embodiments, the flavonoid compound may be isorhamnetin. In certain embodiments, the flavonoid compound may be quercetin.

As used herein, the terms “snake envenomation” and “toxicity induced by snake venom” can be interchangeably used, and refer to a wide range of known toxicities induced by snake venoms, which include, but are not limited to, neurotoxicity, hemotoxicity, vascular toxicity, cardiotoxicity, myotoxicity, and cytotoxicity.

In certain embodiments, the snake envenomation may be caused by a hemotoxic venom. In certain embodiments, the hemotoxic venom may be from Protobothrops spp. Examples of the Protobothrops spp may include, but are not limited to, Protobothrops cornutus, Protobothrops elegans, Protobothrops flavoviridis, Protobothrops himalayanus, Protobothrops jerdonii, Protobothrops kelomohy, Protobothrops mangshanensis, Protobothrops maolanensis, Protobothrops sieversorum, Protobothrops tokarensis, Protobothrops trungkhanhensis, Protobothrops xiangchengensis, and Protobothrops mucrosquamatus. In certain embodiments, the Protobothrops spp. may be Protobothrops mucrosquamatus.

In certain embodiments, the snake envenomation may be caused by a neurotoxic venom. In certain embodiments, the neurotoxic venom may be from Naja spp. Examples of the Naja spp. may include, but are not limited to, Naja annulifera, Naja arabica, Naja ashei, Naja haje, Naja kaouthia, Naja katiensis, Naja mandalayensis, Naja melanoleuca, Naja mossambica, Naja nana, Naja nigricincta, Naja nubiae, Naja oxiana, Naja pallida, Naja philippinensis, Naja sagittifera, Naja samarensis, Naja savannula, Naja senegalensis, Naja siamensis, Naja sputatrix, Naja subfulva, Naja sumatrana, Naja nivea, Naja atra, Naja nigricollis, and Naja naja. In certain embodiments, the Naja spp. may be selected from the group consisting of Naja nivea, Naja atra, Naja nigricollis, Naja naja, and combinations thereof.

According to the disclosure, the pharmaceutical composition may be formulated into a dosage form suitable for parenteral administration, oral administration, or topical administration using technology well known to those skilled in the art.

According to the disclosure, the pharmaceutical composition may further include a pharmaceutically acceptable carrier widely employed in the art of drug-manufacturing. For instance, the pharmaceutically acceptable carrier may include one or more of the following agents: solvents, buffers, emulsifiers, suspending agents, decomposers, disintegrating agents, dispersing agents, binding agents, excipients, stabilizing agents, chelating agents, diluents, gelling agents, preservatives, wetting agents, lubricants, absorption delaying agents, liposomes, and the like. The choice and amount of the aforesaid agents are within the expertise and routine skills of those skilled in the art.

For parenteral administration, the pharmaceutical composition according to the disclosure may be formulated into an injection, e.g., a sterile aqueous solution or a dispersion using technology well known to those skilled in the art. In certain embodiments, the pharmaceutical composition may be administered via intramuscular injection or subcutaneous injection.

According to the disclosure, the dosage form suitable for oral administration may include, but is not limited to, sterile powders, tablets, troches, lozenges, pellets, capsules, dispersible powders or granules, solutions, suspensions, emulsions, syrup, elixir, slurry, and the like.

According to the disclosure, the pharmaceutical composition may be formulated into an external preparation suitable for topical application to the skin using technology well known to those skilled in the art. The external preparation may include, but is not limited to, emulsions, gels, ointments, creams, patches, liniments, powder, aerosols, sprays, lotions, serums, pastes, foams, drops, suspensions, salves, and bandages.

According to the disclosure, the external preparation may be prepared by mixing the pharmaceutical composition with a base well known to those skilled in the art.

According to the disclosure, the base may include one or more of the following additives: water, alcohols, glycol, hydrocarbons (e.g., petroleum jelly and white petrolatum), waxes (e.g., paraffin and yellow wax), preserving agents, antioxidants, surfactants, absorption enhancers, stabilizing agents, gelling agents (e.g., Carbopol® 941, microcrystalline cellulose, and carboxymethylcellulose), active agents, humectants, odor absorbers, fragrances, pH adjusting agents, chelating agents, emulsifiers, occlusive agents, emollients, thickeners, solubilizing agents, penetration enhancers, anti-irritants, colorants, and propellants. The choice and amount of the aforesaid additives are within the expertise and routine skills of those skilled in the art.

As used herein, the terms “administration” and “administrating” can be interchangeably used, and mean introducing, providing or delivering the abovementioned pharmaceutical composition to a subject showing sign(s) of snake envenomation by any suitable routes to perform its intended function.

As used herein, the term “alleviating” or “alleviation” refers to at least partially reducing, ameliorating, relieving, controlling, treating or eliminating one or more clinical signs of a disease or disorder; and lowering, delaying, stopping or reversing the progression of severity regarding the condition or symptom being treated and preventing or decreasing the likelihood or probability thereof.

As used herein, the term “subject” refers to any mammal of interest, such as humans, monkeys, cows, sheep, horses, pigs, goats, dogs, cats, mice, and rats. In certain embodiments, the subject is a human.

The dose and frequency of administration of the pharmaceutical composition of the disclosure may vary depending on the following factors: the severity of the illness or disorder to be treated, routes of administration, and age, physical condition and response of the subject to be treated. In general, the pharmaceutical composition may be administered in a single dose or in several doses.

The disclosure will be further described by way of the following examples. However, it should be understood that the following examples are intended solely for the purpose of illustration and should not be construed as limiting the disclosure in practice.

### EXAMPLES

### General Experimental Materials:

### 1. Experimental Mice:

Institute of Cancer Research (ICR) mice (5 weeks old, with a body weight of approximately 10 g to 15 g) used in the following experiments were purchased from BioLASCO Taiwan Co., Ltd. All the experiments mice were housed in an animal room under the following laboratory conditions: an alternating 12-hour light and 12-hour dark cycle, a temperature maintained at 24° C., and a relative humidity maintained at 65%. Furthermore, water and food were provided at libitum for all the experimental mice. All experimental procedures involving the experimental mice were in compliance with the guidelines of the Institutional Animal Care and Use Committees of National Tsing Hua University.

### 2. Flavonoid Compounds:

Isorhamnetin, quercetin, and apigenin, which are three kinds of flavonoid compounds, used in the following experiments were purchased from Sigma-Aldrich.

### Example 1. Evaluation of the Efficacy of Flavonoid Compounds Against Death Caused by Snake Envenomation

### Experimental Materials:

### A. Snake Venoms

The snake venoms from different snake species used in this example and their corresponding median lethal doses (LD₅₀) for the ICR mice are shown in Table 1 below.

TABLE 1

| | | | LD₅₀ |
|---|---|---|---|
| Type of venom | Snake species | Source | (μg/g) |
| Hemotoxic venom | Protobothrops | Tainan Snake | 2.2 |
| | mucrosquamatus | King Zoo | |
| Neurotoxic venom | Naja atra | | 0.5 |
| | Naja nivea | Latoxan | 1.11 |
| | Naja nigricollis | | 0.9 |
| | Naja naja | | 0.45 |

### Experimental Procedures and Results:

### A. Efficacy of Flavonoid Compounds Against Death Caused by Hemotoxic Venom

The ICR mice were randomly divided into four groups, which included a pathological control group, two experimental groups (i.e., experimental groups 1 and 2), and one comparative group (n=7 mice in each group). Next, a respective one of the isorhamnetin, the quercetin, and the apigenin was mixed with a venom of Protobothrops mucrosquamatus in a volume ratio of 1:20, followed by incubation at 37° C. for 30 minutes, thereby forming a respective mixture solution. Afterward, the respective ICR mouse of each of the experimental groups 1 and 2 and the comparative group was intradermally injected with a suitable amount of the mixture solution as shown in Table 2 below. In addition, the respective ICR mouse in the pathological control group was intradermally injected with the venom of Protobothrops mucrosquamatus. The treating agent and the dose thereof for each group are summarized in Table 2 below.

TABLE 2

| | Treating agent | |
|---|---|---|
| | Venom of Protobothrops | |
| Group | mucrosquamatus | Flavonoid compound |
| Pathological control | Dose: 2.5 × LD₅₀ per | — |
| group | mouse | |
| Experimental group 1 | | Isorhamnetin |
| | | (dose: 50 nmole per |
| | | mouse) |
| Experimental group 2 | | Quercetin |
| | | (dose: 50 nmole per |
| | | mouse) |
| Comparative group | | Apigenin |
| | | (dose: 50 nmole per |
| | | mouse) |

Subsequently, the survival number of the ICR mice in each group was observed and recorded at every hour until 10 hours after injection with the treating agent. The viability percentage (%) of the ICR mice in each group was calculated using the following Equation (1):

A=(B/7)×100 (1)

where A=viability percentage (%)

B=survival number of the ICR mice at every hour until 10 hours after injection with treating agent

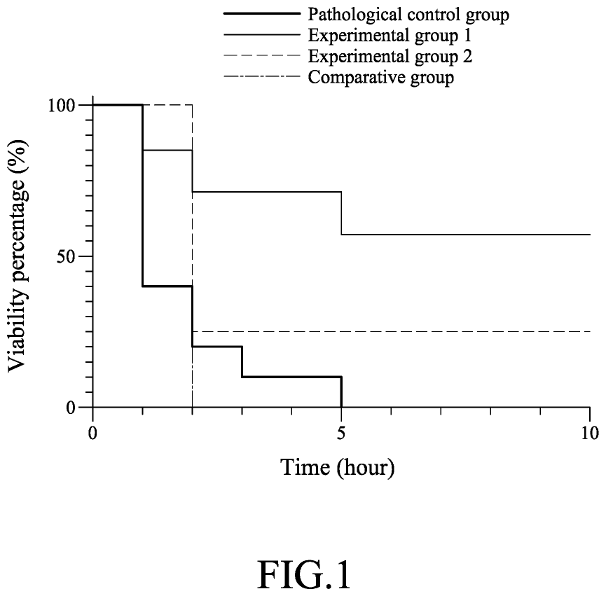

Referring to FIG. 1, the viability percentage determined in the pathological control group dropped sharply at the first hour after injection with the treating agent, and continually fell to 0% at the fifth hour after injection with the treating agent. Moreover, the viability percentage of the ICR mice in the comparative group dramatically dropped to 0% at the second hour after injection with the treating agent, while the viability percentage of the ICR mice in the experimental group 2 was 25% at the second hour after injection with the treating agent, and remained at the same level until the tenth hour after injection with the treating agent. In addition, the viability percentage of the ICR mice in the experimental group 1 exceeded 70% at the second hour after injection with the treating agent, and even remained above 55% at the tenth hour after injection with the treating agent. These results indicate that both quercetin and isorhamnetin are effective against death caused by a hemotoxic venom and hence are capable of prolonging survival time, while apigenin, despite having a similar structure to those of quercetin and isorhamnetin, not only does not exhibit such effects (i.e., against death and prolonging survival time), but instead, may accelerate death.

### B. Efficacy of Isorhamnetin Against Death Caused by Neurotoxic Venom

Based on the results in Section A of this example, which show that isorhamnetin has the best outcome against death caused by the hemotoxic venom (i.e., the venom of Protobothrops mucrosquamatus) among the other flavonoid compounds, the applicants would like to further study the efficacy of isorhamnetin against death caused by a neurotoxic venom.

First, the ICR mice were randomly divided into eight groups, including four pathological control groups (i.e., pathological control groups 3 to 6) and four experimental groups (i.e., experimental groups 3 to 6) (n=7 mice in each group). Next, the isorhamnetin was mixed with a respective one of a venom of Naja nigricollis, a venom of Naja nivea, a venom of Naja naja, and a venom of Naja atra in a volume ratio of 1:20, followed by incubation at 37° C. for 30 minutes, thereby forming a respective mixture solution. Afterward, the respective ICR mouse of each of the experimental groups 3 to 6 was intradermally injected with a suitable amount of the mixture solution as shown in Table 3 below. In addition, the respective ICR mouse in each of the pathological control groups 3 to 6 was intradermally injected with the venom of Naja nigricollis, the venom of Naja nivea, the venom of Naja naja, and the venom of Naja atra, respectively. The treating agent and the dose thereof for each group are summarized in Table 3 below.

TABLE 3

| Group | Treating agent | |
|---|---|---|
| | Venom of Naja nigricollis | Isorhamnetin |
| Pathological control | Dose: 2.5 × LD₅₀ per | — |
| group 3 | mouse | |
| Experimental group 3 | | Dose: 50 nmole (per |
| | | mouse) |
| | Venom of Naja nivea | Isorhamnetin |
| Pathological control | Dose: 2.5 × LD₅₀ per | — |
| group 4 | mouse | |
| Experimental group 4 | | Dose: 50 nmole (per |
| | | mouse) |
| | Venom of Naja naja | Isorhamnetin |
| Pathological control | Dose: 2.5 × LD₅₀ per | — |
| group 5 | mouse | |
| Experimental group 5 | | Dose: 50 nmole (per |
| | | mouse) |
| | Venom of Naja atra | Isorhamnetin |
| Pathological control | Dose: 2.5 × LD₅₀ per | — |
| group 6 | mouse | |
| Experimental group 6 | | Dose: 50 nmole (per |
| | | mouse) |

Subsequently, the survival number of the ICR mice in each group was observed and recorded at every hour until 14 hours after injection with the treating agent. The viability percentage (%) of the ICR mice in each group was calculated using the Equation (1).

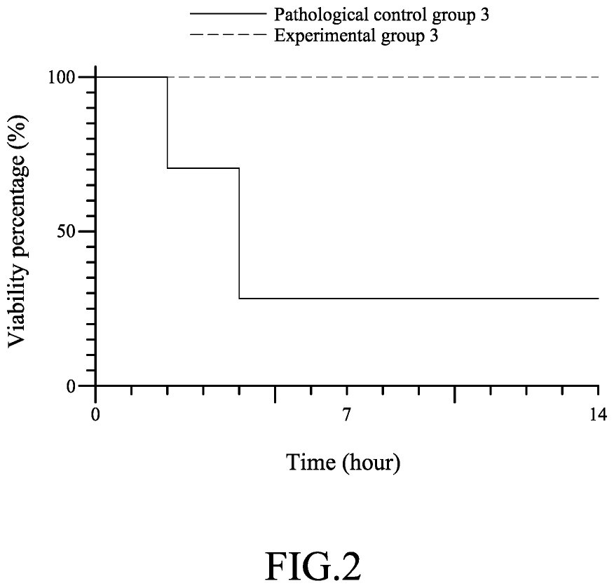

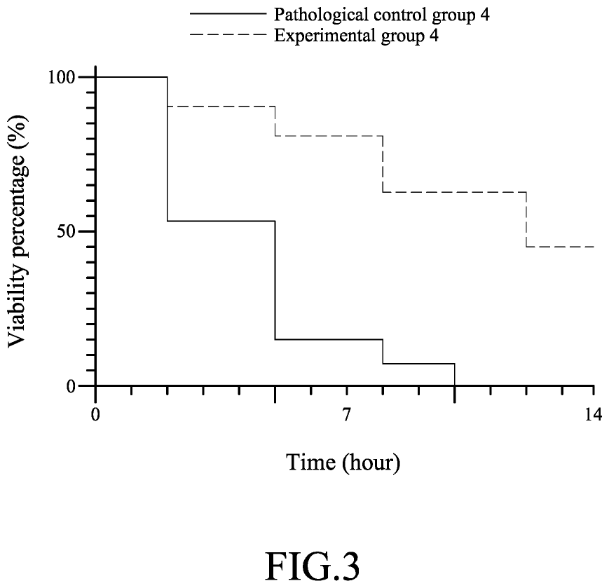

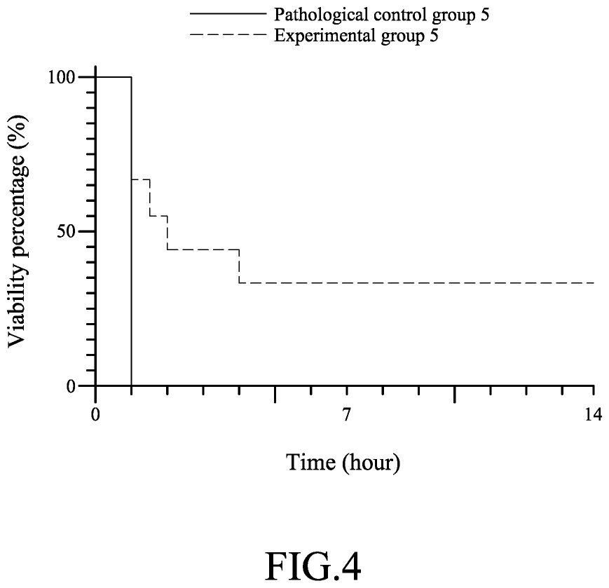

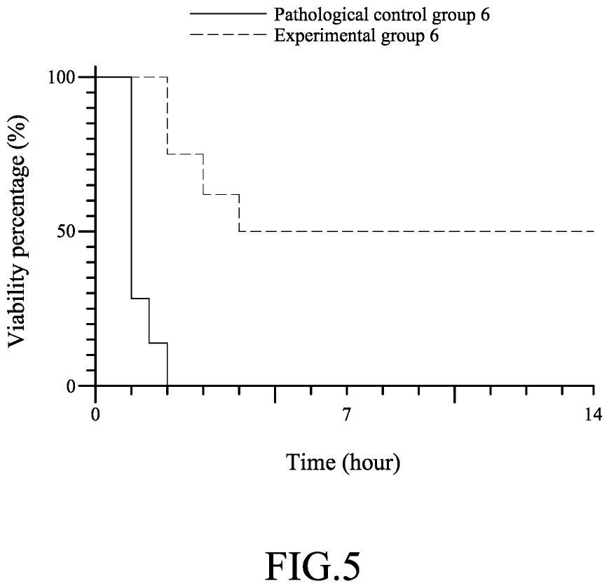

Referring to FIG. 2, the viability percentage determined in the pathological control group 3 had already dropped to 30% at the fourth hour after injection with the treating agent, while the viability percentage determined in the experimental group 3 maintained at 100% until the fourteenth hour after injection with the treating agent. Referring to FIG. 3, the viability percentage determined in the pathological control group 4 had already dropped below 20% at the fifth hour after injection with the treating agent, and kept decreasing to 0% at the tenth hour after injection with the treating agent, while the viability percentage determined in the experimental group 4 was higher than 65% at the tenth hour after injection with the treating agent, and remained at above 45% at the fourteenth hour after injection with the treating agent. Referring to FIG. 4, the viability percentage determined in the pathological control group 5 had already dropped to 0% at the first hour after injection with the treating agent, while the viability percentage determined in the experimental group 5 was higher than 65% at the first hour after injection with the treating agent, and remained at above 30% at the fourteenth hour after injection with the treating agent. Referring the FIG. 5, the viability percentage determined in the pathological control group 6 had already dropped to 0% at the second hour after injection with the treating agent, while the viability percentage determined in the experimental group 6 was higher than 75% at the second hour after injection with the treating agent, and still remained at above 50% at the fourteenth hour after injection with the treating agent.

These results show that isorhamnetin is effective against death caused by a neurotoxic venom and hence is capable of prolonging survival time.

C. Efficacy of Isorhamnetin in Alleviating Toxicity Arising from Neurotoxic Venom

To closely simulate conditions of an emergency treatment in which the subject was a victim of a venomous snake bite, in this application, the ICR mice were injected with the venom of Naja nivea and the isorhamnetin separately (with a time interval therebetween) instead of simultaneously (i.e., mixed in advance for injection).

First, the ICR mice were randomly divided into two groups, including a pathological control group (i.e., pathological control group 7) and an experimental group (i.e., experimental group 7) (n=7 mice in each group). Next, the respective ICR mouse in each group was intradermally injected with a suitable amount of the venom of Naja nivea (dose: 2.5×LD₅₀ per mouse). After 30 minutes, the respective ICR mouse in the experimental group was intradermally injected again with a suitable amount of the isorhamnetin (dose: 50 nmole per mouse).

Subsequently, the survival number of the ICR mice in each group was observed and recorded at every hour until 48 hours after injection with the treating agent. The viability percentage (%) of the ICR mice in each group was calculated using the Equation (1).

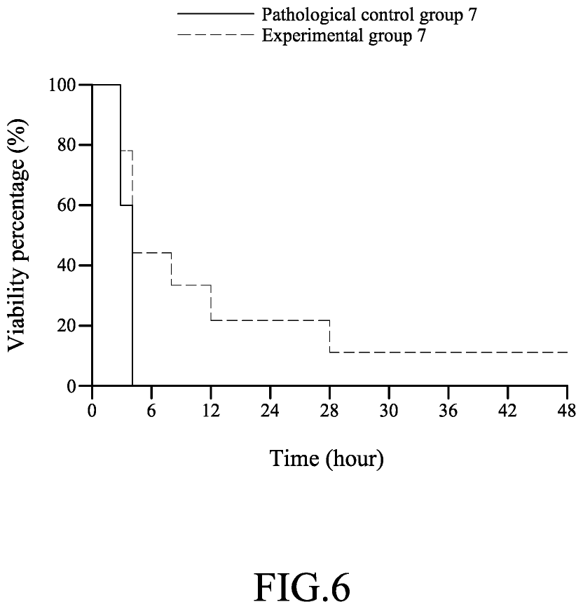

Referring to FIG. 6, the viability percentage determined in the pathological control group 7 had already dropped to 0% at the fifth hour after injection with the treating agent, while the viability percentage determined in the experimental group 7 was higher than 40% at the same time, and remained higher than 20% from the twelfth hour to the 28th hour after injection with the treating agent. Furthermore, some of the ICR mice in the experimental group 7 could even survive until the 48th hour after injection with the treating agent. These results show that administration of isorhamnetin to a subject who is affected by a snake venom is effective against death caused thereby and hence is capable of prolonging survival time.

### Example 2. Evaluation of the Efficacy of Flavonoid Compounds in Alleviating Cutaneous Ulceration Caused by Snake Venom Proteins

For further study of the efficacy of isorhamnetin and quercetin in alleviating cutaneous ulceration caused by snake venom proteins, the applicants conducted experiments using a snake venom protein mixture containing snake venom proteins having a molecular weight of not lower than 43 kD and a cardiotoxin that might cause tissue necrosis, so as to avoid sudden death of the ICR mice due to other toxins that might have interfered with the observation.

### Experimental Materials:

### 1. Preparation of Snake Venom Protein Mixture

First, cardiotoxins were isolated from a suitable amount of the venom of Naja atra with reference to the methods descried in SC Sue et al., (2001), Biochemistry, 40, 12782-12794. In addition, another suitable amount of the venom of Naja atra was dissolved in a buffer solution (pH 6.4) containing 50 mM of phosphate and 150 mM of sodium chloride, so as to form a mixture, followed by subjecting the mixture to gel filtration chromatography using a Superdex® 75 column (GE Healthcare Life Sciences), thereby collecting high molecular weight proteins that had a molecular weight of not lower than 43 kD. Next, the cardiotoxins and the high molecular weight proteins were mixed, so as to obtain a snake venom protein mixture containing 0.5 mg/mL of the cardiotoxins and 0.025 mg/mL of the high molecular weight proteins.

### Experimental Procedures and Results:

### A. Efficacy of Flavonoid Compounds Against Cutaneous Ulceration Caused by Snake Venom Proteins Via Intradermal Injection

First, the ICR mice were randomly divided into three groups, including a pathological control group and two experimental groups (i.e., experimental groups 1 and 2) (n=7 mice in each group). Next, a respective one of the quercetin and isorhamnetin was mixed with a suitable amount of the snake venom protein mixture in a volume ratio of 1:20, followed by incubation at 37° C. for minutes, thereby forming a respective mixture solution. Afterward, the respective ICR mouse of the experimental groups 1 and 2 was intradermally injected with a suitable amount of the mixture solution as shown in Table 4 below. In addition, the respective ICR mouse in the pathological control group was intradermally injected with the snake venom protein mixture. The treating agent and the dose thereof for each group are summarized in Table 4 below.

TABLE 4

| | Treating agent | |
|---|---|---|
| | Snake venom protein | |
| Group | mixture | Flavonoid compound |
| Pathological control | Dose: 0.1 mL per mouse | — |
| group | | |
| Experimental group 1 | | Quercetin |
| | | (dose: 50 nmole per |
| | | mouse) |
| Experimental group 2 | | Isorhamnetin |
| | | (dose: 50 nmole per |
| | | mouse) |

At the 24^{th} hour after injection with the treating agent, cutaneous ulcers occurring in the ICR mice of each group were photographed and the area thereof was analyzed using ImageJ software.

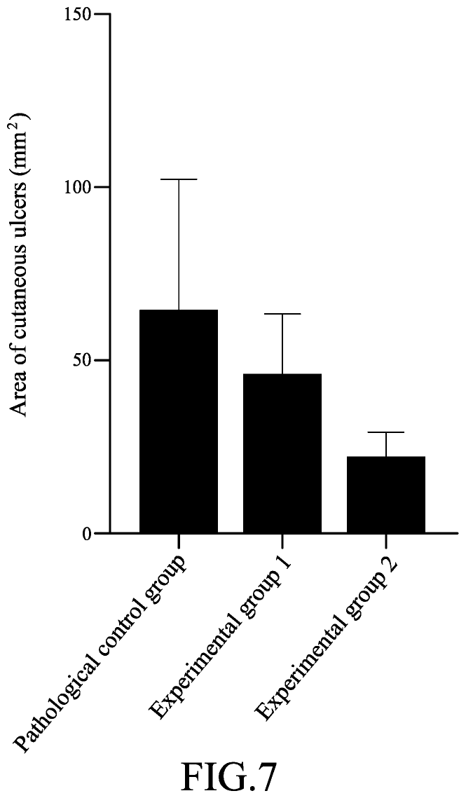

Referring to FIG. 7, the areas of the cutaneous ulcers determined in the experimental group 1 and 2 were significantly reduced compared to that of the pathological control group, in which the area of the cutaneous ulcers determined in the experimental group 2 had the highest degree of reduction.

### B. Efficacy of Isorhamnetin Against Cutaneous Ulceration Caused by Snake Venom Proteins Via Topical Administration

For further evaluating whether or not a similar result, as that of the experimental group 2 in Section A of this example, could be obtained when the isorhamnetin was applied via topical administration immediately after a subject suffered from a venomous snake bite, the following experiments were conducted.

First, 3.1 mg of the isorhamnetin was mixed with 1 mL of petroleum jelly in a melted state at 45° C., thereby obtaining an isorhamnetin ointment. Afterward, the ICR mice were randomly divided into two groups, including a pathological control group (pathological control group 3) and an experimental group (i.e., experimental group 3) (n=7 mice in each group). Next, the respective ICR mouse of each group was intradermally injected with a suitable amount of the snake venom protein mixture (0.1 mL per mouse). After that, an appropriate amount of the isorhamnetin ointment was instantly applied onto the injection area of a respective one of the ICR mice in the experimental group 3 (0.2 g/cm², once a day); as for the ICR mice in the pathological control group 3, the same amount of petroleum jelly was applied onto each mouse thereof.

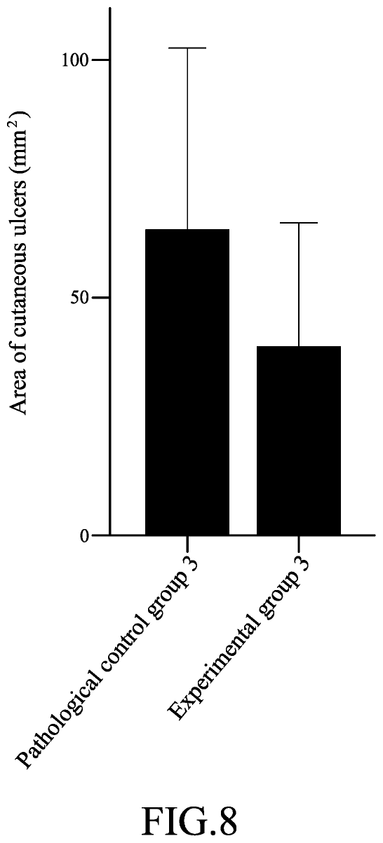

Referring to FIG. 8, the area of cutaneous ulcers in the experimental group was significantly reduced compared to that of the pathological control group.

Moreover, the applicants further determined a median effective dose (ED₅₀) of each of isorhamnetin and quercetin for reducing an area of a cutaneous ulcer caused by snake venom proteins. The results of the determination reveal that the median effective doses of isorhamnetin and quercetin are 0.2 mM and 1.2 mM, respectively, if being administered via intradermal injection, and the median effective dose of isorhamnetin is 0.6 mM if being done via topical administration. The results show that both isorhamnetin and quercetin are effective against tissue necrosis caused by snake venom proteins through different administration routes.

In sum, it can be seen that isorhamnetin and quercetin can not only resist death caused by snake venoms but also alleviate tissue necrosis arising therefrom, and hence are expected to be used against snake venoms or snake envenomation.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method against snake envenomation, comprising administering to a subject in need thereof a pharmaceutical composition containing a flavonoid compound, the flavonoid compound being selected from the group consisting of isorhamnetin, quercetin, and a combination thereof.

2. The method as claimed in claim 1, wherein the snake envenomation is caused by a hemotoxic venom.

3. The method as claimed in claim 2, wherein the hemotoxic venom is from Protobothrops spp.

4. The method as claimed in claim 3, wherein the Protobothrops spp. is Protobothrops mucrosquamatus.

5. The method as claimed in claim 1, wherein the snake envenomation is caused by a neurotoxic venom.

6. The method as claimed in claim 5, wherein the neurotoxic venom is from Naja spp.

7. The method as claimed in claim 6, wherein the Naja spp. is selected from the group consisting of Naja nivea, Naja atra, Naja nigricollis, Naja naja, and combinations thereof.

8. The method as claimed in claim 1, wherein the pharmaceutical composition is in a dosage form selected from the group consisting of a parenteral dosage form, an oral dosage form, and a topical dosage form.
